(19) United States
(12) Patent Application Publication
(10) Pub. No.: US 2021/0249130 A1
(43) Pub. Date: Aug. 12, 2021

(54) INFORMATION PROCESSING DEVICE, INFORMATION PROCESSING METHOD, AND SYSTEM

(71) Applicant: TOYOTA JIDOSHA KABUSHIKI KAISHA, Toyota-shi (JP)

(72) Inventors: Mitsuyoshi OHNO, Miyoshi-shi (JP); Daiki KUBO, Toyota-shi (JP); Yosuke NOZAKI, Toyota-shi (JP); Tsukasa NAKANISHI, Nagoya-shi (JP); Hironori AOYAMA, Toyota-shi (JP); Seiji YOGO, Nagoya-shi (JP); Tae SUGIMURA, Miyoshi-shi (JP); Yasuhiro KOBATAKE, Nagoya-shi (JP); Takeshi YAMADA, Anjo-shi (JP); Ryo SATO, Nisshin-shi (JP); Sayaka NINOYU, Toyota-shi (JP)

(73) Assignee: TOYOTA JIDOSHA KABUSHIKI KAISHA, Toyota-shi (JP)

(21) Appl. No.: 17/130,873

(22) Filed: Dec. 22, 2020

(30) Foreign Application Priority Data
Feb. 6, 2020 (JP) 2020-018852

(51) Int. Cl. G16H 40/67; G16H 40/20; G05D 1/02

(52) U.S. Cl. CPC G16H 40/67; G16H 40/20; G05D 2201/0212; G05D 2201/0206; G05D 1/0225

(57) ABSTRACT

A control unit is provided that executes: acquiring a first request that is a request based on information regarding a physical condition of a user using a bed unit that functions as a bed for the user, and that is a request to move the bed unit from a house of the user to a hospital; when having acquired the first request, selecting a first traveling unit that is a traveling unit that moves the bed unit from the house to the hospital and that is a traveling unit located within a predetermined distance from the bed unit; and generating a command for the first traveling unit such that the first traveling unit carries the bed unit from the house to the hospital.

### INCORPORATION BY REFERENCE

The disclosure of Japanese Patent Application No. 2020-018852 filed on Feb. 6, 2020 including the specification, drawings and abstract is incorporated herein by reference in its entirety.

### BACKGROUND

### 1. Technical Field

The present disclosure relates to an information processing device, an information processing method, and a system.

### 2. Description of Related Art

It is known that an upper unit of a vertically separable vehicle constitutes a part of a building (see, for example, WO 2018/230720).

### SUMMARY

In the case where the upper unit is used as a bed, it may be difficult to quickly call the lower unit when the physical condition of the user deteriorates. An object of the present disclosure is to carry a user to the hospital together with the bed.

One aspect of the present disclosure provides an information processing device including a control device. The control device executes: acquiring a first request that is a request based on information regarding a physical condition of a user using a bed unit that functions as a bed for the user, and that is a request to move the bed unit from a house of the user to a hospital; when having acquired the first request, selecting a first traveling unit that is a traveling unit that moves the bed unit from the house to the hospital and that is a traveling unit located within a predetermined distance from the bed unit; and generating a command for the first traveling unit such that the first traveling unit carries the bed unit from the house to the hospital.

One aspect of the present disclosure provides an information processing method. The information processing method causes a computer to execute: acquiring a first request that is a request based on information regarding a physical condition of a user using a bed unit that functions as a bed for the user, and that is a request to move the bed unit from a house of the user to a hospital; when having acquired the first request, selecting a first traveling unit that is a traveling unit that moves the bed unit from the house to the hospital and that is a traveling unit located within a predetermined distance from the bed unit; and generating a command for the first traveling unit such that the first traveling unit carries the bed unit from the house to the hospital.

One aspect of the present disclosure provides a system including: a bed unit that functions as a bed for a user; a traveling unit that is connected to the bed unit and moves the bed unit; and a server having a control unit that controls the traveling unit. The control unit executes: acquiring a first request that is a request based on information regarding a physical condition of the user using the bed unit and that is a request to move the bed unit from the house of the user to the hospital; when having acquired the first request, selecting a first traveling unit that is a traveling unit that moves the bed unit from the house to the hospital and that is a traveling unit located within a predetermined distance from the bed unit; and generating a command for the first traveling unit such that the first traveling unit carries the bed unit from the house to the hospital.

Another aspect of the present disclosure provides a program for causing a computer to execute the above-described information processing method, or a storage medium that non-temporarily stores the program.

According to the present disclosure, a user can be carried to a hospital together with a bed.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram showing a schematic configuration of an autonomous driving system according to an embodiment;

FIG. 2 is a diagram showing a schematic configuration when a vehicle is connected to a house;

FIG. 3 is a diagram showing a schematic configuration after a traveling unit goes away leaving a bed unit at the house;

FIG. 4 is a block diagram schematically showing an example of respective configurations of the bed unit, the traveling unit, and the server that constitute the autonomous driving system according to the present embodiment;

FIG. 5 is a diagram showing an example of a functional configuration of the server;

FIG. 6 is a diagram illustrating a table configuration of user information;

FIG. 7 is a diagram illustrating a table configuration of bed unit information.

FIG. 8 is a diagram illustrating a table configuration of traveling unit information;

FIG. 9 is a diagram showing an example of a functional configuration of the traveling unit;

FIG. 10 is a diagram showing an example of a functional configuration of the bed unit;

FIG. 11 is a sequence diagram showing processes of the autonomous driving system;

FIG. 12 is an example of a flowchart illustrating processes in the server according to the present embodiment;

FIG. 13 is an example of a flowchart illustrating processes in the traveling unit according to the present embodiment; and

FIG. 14 is an example of a flowchart illustrating processes of transmitting a use request according to the present embodiment.

### DETAILED DESCRIPTION OF EMBODIMENTS

A traveling unit controlled by an information processing device, which is one aspect of the present disclosure, is a vehicle that autonomously travels based on an operation command, for example. The traveling unit can be connected to a bed unit, and can carry the bed unit with the bed unit connected thereto. The bed unit has such facilities that a user can use the bed unit as a hospital room. The traveling unit can also travel even when the bed unit is not connected thereto. Thus, the traveling unit can leave the place where the bed unit is placed after placing the bed unit.

A control unit acquires a first request that is a request based on information regarding a physical condition of a user using a bed unit that functions as a bed for the user, and that is a request to move the bed unit from a house of the user to a hospital. The information regarding the physical condition of the user is, for example, information indicating that the physical condition of the user has deteriorated to such an extent that it is necessary to transport the user to the hospital. Here, the user using the bed unit may have difficulty walking. It may take time for such a user to move to the hospital. Therefore, the control unit provides a command to the traveling unit such that the traveling unit carries the bed unit with the bed unit carrying the user. The bed unit may have a structure that can be united with the house of the user.

When having acquired the first request, the control unit selects a first traveling unit that is a traveling unit that moves the bed unit from the house to the hospital and that is a traveling unit located within a predetermined distance from the bed unit. That is, the first traveling unit for carrying the bed unit is selected from traveling units located within a predetermined distance from the bed unit. The predetermined distance is set, for example, so that the time required for the traveling unit to arrive at the current position of the bed unit falls within an allowable range. By selecting such a first traveling unit, it is possible to suppress an increase in the time required to carry the user to the hospital. In addition, since the traveling unit need to be connected to the bed unit only when carrying the bed unit, the traveling unit does not need to be always waiting at the house of the user. Thus, the traveling unit can also carry another bed unit carrying another user, which can increase the operating rate of the traveling unit.

Further, the control unit generates a command for the first traveling unit such that the first traveling unit carries the bed unit from the house to the hospital. The first traveling unit travels based on this command, so that the user can be carried to the hospital together with the bed unit.

Hereinafter, embodiments of the present disclosure will be described with reference to the drawings. The configurations of the following embodiments are illustrative, and the present disclosure is not limited to the configurations of the embodiments. Further, the following embodiments can be combined as much as possible.

### First Embodiment

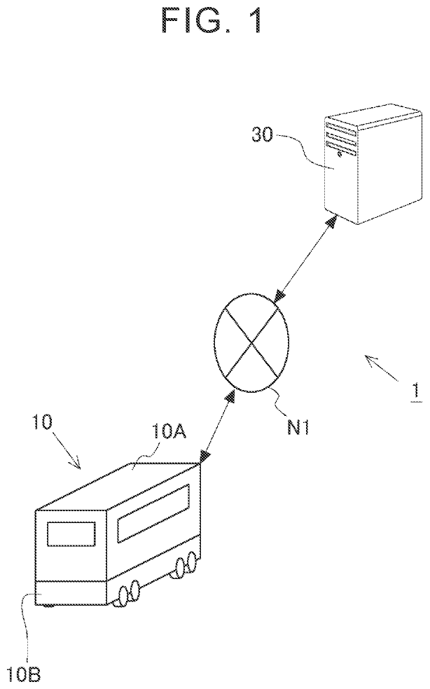

FIG. 1 is a diagram showing a schematic configuration of an autonomous driving system 1 according to an embodiment. The autonomous driving system 1 includes, for example, a vehicle 10 and a server 30. The vehicle 10 includes, for example, a bed unit 10A and a traveling unit 10B. The bed unit 10A and the traveling unit 10B can be separated. Also, the bed unit 10A can be connected to a different traveling unit 10B. Further, the traveling unit B can be connected to a different bed unit 10A. The numbers of the bed units 10A and the traveling units 10B are not limited to one illustrated in FIG. 1 and may be two or more. The traveling unit 10B is a moving body that is capable of autonomous traveling based on an operation command generated by the server 30.

The autonomous driving system 1 shown in FIG. 1 is a system in which the server 30 selects the traveling unit 10B for carrying the bed unit 10A when a request to move the bed unit 10A (hereinafter, also referred to as a use request) is made. The request to move the bed unit 10A is transmitted from the bed unit 10A to the server 30. For example, it is considered that a request to move the bed unit 10A is made when the user using the bed unit 10A presses a button or when a sensor for sensing the user's physical condition or seriousness of the user's condition detects deterioration of the user's physical condition or the like.

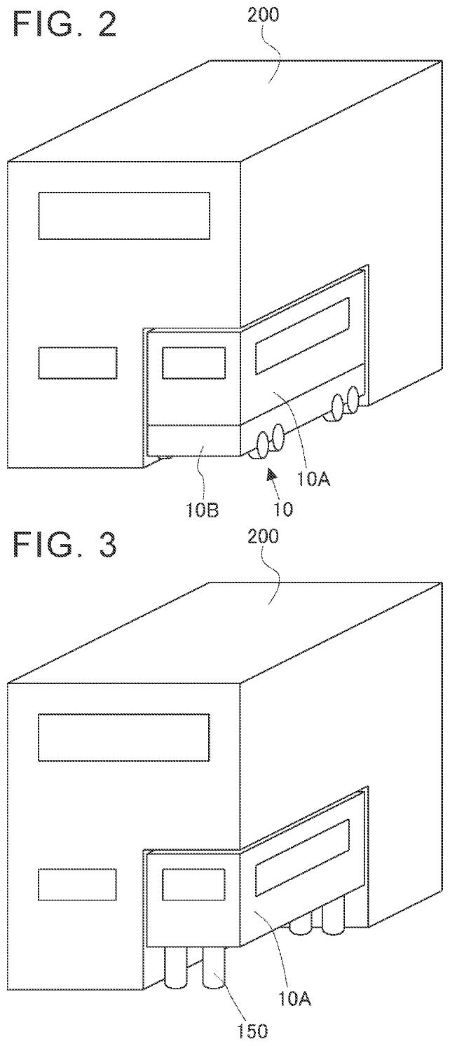

The bed unit 10A serves as a bed for the user. The bed may be a hospital room or a nursing room. Here, FIG. 2 is a diagram showing a schematic configuration when the vehicle 10 is connected to a house 200. FIG. 3 is a diagram showing a schematic configuration after the traveling unit 10B goes away leaving the bed unit 10A at the house 200. The bed unit 10A includes, for example, a bed. The traveling unit 10B can be connected to the bed unit 10A and can carry the bed unit 10A with the user in the bed unit 10A. The bed unit 10A becomes movable when the traveling unit 10B is connected thereto. Also, the bed unit 10A can be used as a hospital room while being connected to the house 200, even when the bed unit 10A is disconnected from the traveling unit 10B. Further, the traveling unit 10B can move even when the bed unit 10A is not connected. In FIG. 1, FIG. 2, and FIG. 3, the bed unit 10A and the traveling unit 10B can be separated vertically, but the disclosure is not limited to this, and may be separated to the front and the rear along the traveling direction, for example. Further, the bed unit 10A can be a hospital room for the user in the hospital after moving from the house 200 to the hospital. That is, the hospital has the same structure as the house 200, and when the traveling unit 10B disconnects the bed unit 10A at the hospital, the bed unit 10A functions as a hospital room as it is.

When the server 30 receives from the bed unit 10A a request to carry the bed unit 10A to the hospital, the server 30 selects the traveling unit 10B for carrying the bed unit 10A connected to the house 200 to the hospital. Then, the server 30 transmits a command to the traveling unit 10B that is selected to carry the bed unit 10A (first traveling unit) to the hospital. The command may include, for example, information regarding locations of the house 200 and the hospital, or information regarding a route on which the traveling unit 10B moves to the hospital via the house 200. In addition, the command includes, for example, a command to connect the bed unit 10A to carry the bed unit 10A to the hospital and a command to disconnect the bed unit 10A at the hospital. The traveling unit 10B that has disconnected the bed unit 10A at the hospital can move to another place by autonomous traveling. Thereby, the traveling unit 10B can carry another bed unit 10A.

The bed unit 10A, the traveling unit 10B, and the server 30 are mutually connected by a network N1. The network N1 is, for example, a world-wide public communication network such as the Internet, and a wide area network (WAN) or other communication networks may be adopted. In addition, the network N1 may include a telephone communication network such as a mobile phone network and a wireless communication network such as Wi-Fi (registered trademark).

### Hardware Configuration

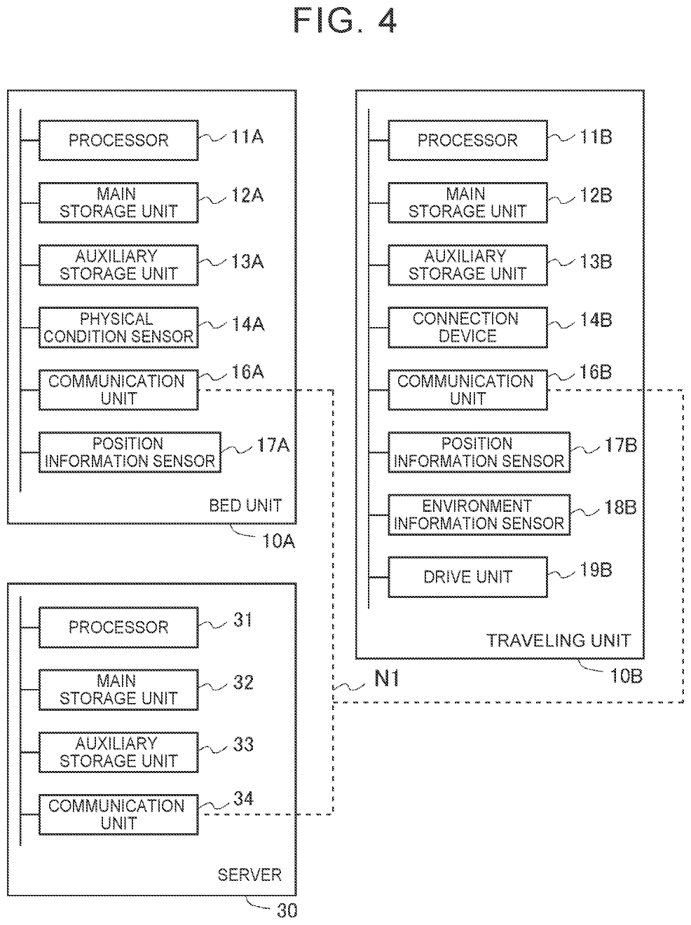

The hardware configurations of the bed unit 10A, the traveling unit 10B, and the server 30 will be described based on FIG. 4. FIG. 4 is a block diagram schematically showing an example of respective configurations of the bed unit 10A, the traveling unit 10B, and the server 30 that constitute the autonomous driving system 1 according to the present embodiment.

The server 30 has a general computer configuration. The server 30 includes a processor 31, a main storage unit 32, an auxiliary storage unit 33, and a communication unit 34. These are connected to each other by a bus.

The processor 31 is a central processing unit (CPU), a digital signal processor (DSP), or the like. The processor 31 controls the server 30 and performs various information processing calculations. The processor 31 is an example of a “control unit”. The main storage unit 32 is a read only memory (ROM), a random access memory (RAM), or the like. The auxiliary storage unit 33 is an erasable programmable ROM (EPROM), a hard disk drive (HDD), a removable medium, or the like. The auxiliary storage unit 33 stores an operating system (OS), various kinds of programs, various kinds of tables, and the like. The processor 31 loads the program stored in the auxiliary storage unit 33 into the work area of the main storage unit 32 and executes the program. Through execution of the program, each component is controlled. As a result, the server 30 realizes the function that matches the predetermined purpose. The main storage unit 32 and the auxiliary storage unit 33 are computer-readable recording media. The server 30 may be a single computer or may include a plurality of computers linked together. Further, the information stored in the auxiliary storage unit 33 may be stored in the main storage unit 32. Further, the information stored in the main storage unit 32 may be stored in the auxiliary storage unit 33.

The communication unit 34 is means for communicating with the bed unit 10A and the traveling unit 10B via the network N1. The communication unit 34 is, for example, a local area network (LAN) interface board or a wireless communication circuit for wireless communication. The LAN interface board and the wireless communication circuit are connected to the network N1.

The series of processes executed by the server 30 can be executed by hardware or software. The hardware configuration of the server 30 is not limited to that shown in FIG. 2. Moreover, part or all of the configuration of the server 30 may be mounted on the traveling unit 10B.

Next, the traveling unit 10B will be described. The traveling unit 10B includes a processor 11B, a main storage unit 12B, an auxiliary storage unit 13B, a connection device 14B, a communication unit 16B, a position information sensor 17B, an environment information sensor 18B, and a drive unit 19B. These are connected to each other by a bus. The processor 11B, the main storage unit 12B, the auxiliary storage unit 13B, and the communication unit 16B are similar to the processor 31, the main storage unit 32, the auxiliary storage unit 33, and the communication unit 34 of the server 30, and thus the description thereof will be omitted.

The connection device 14B is a device that connects and disconnects the bed unit 10A and the traveling unit 10B based on a control command generated by the processor 11B. The connection device 14B has, for example, a mechanism for connecting the bed unit 10A to the traveling unit 10B. The connection device 14B has, for example, an electromagnet, a slope, a rail, a crane, or the like. Further, the connection device 14B has an actuator or the like, and the actuator or the like may be activated when the bed unit 10A and the traveling unit 10B are connected or disconnected. In addition, the connection method between the bed unit 10A and the traveling unit 10B is not limited. The connection device 14B may be provided in the bed unit 10A instead of the traveling unit 10B.

The communication unit 16B is communication means for connecting the traveling unit 10B to the network N1. The communication unit 16B is a circuit for communicating with other devices (the server 30 and the like) via the network N1 using a mobile communication service (for example, a telephone communication network such as 5th Generation (5G), 4th Generation (4G), 3rd Generation (3G), and long term evolution (LTE), or wireless communication network such as Wi-Fi (registered trademark).

The position information sensor 17B acquires position information (for example, latitude and longitude) of the traveling unit 10B at a predetermined cycle. The position information sensor 17B is, for example, a Global Positioning System (GPS) receiving unit, a wireless communication unit, or the like. The information acquired by the position information sensor 17B is recorded in, for example, the auxiliary storage unit 13B or the like and transmitted to the server 30.

The environment information sensor 18B is means for sensing the state of the traveling unit 10B or sensing the periphery of the traveling unit 10B. Examples of the sensor for sensing the state of the traveling unit 10B include an acceleration sensor, a speed sensor, or an azimuth sensor. Examples of the sensor for sensing the periphery of the traveling unit 10B include a stereo camera, a laser scanner, a light detection and ranging (LIDAR), a radar, or the like.

The drive unit 19B causes the traveling unit 10B to travel based on the control command generated by the processor 11B. The drive unit 19B is configured to include, for example, a motor and an inverter for driving vehicle wheels of the traveling unit 10B, a brake, a steering mechanism, or the like. The autonomous traveling of the traveling unit 10B is realized when the motor, the brake, or the like is driven in accordance with the control command.

Next, the bed unit 10A will be described. The bed unit 10A includes a processor 11A, a main storage unit 12A, an auxiliary storage unit 13A, a physical condition sensor 14A, a communication unit 16A, and a position information sensor 17A. These are connected to each other by a bus. The processor 11A, the main storage unit 12A, the auxiliary storage unit 13A, the communication unit 16A, and the position information sensor 17A of the bed unit 10A are similar to the processor 11B, the main storage unit 12B, the auxiliary storage unit 13B, the communication unit 16B, and the position information sensor 17B of the traveling unit 10B, and thus the description thereof will be omitted.

The physical condition sensor 14A is a sensor that detects a physical quantity related to the physical condition of the user, and is, for example, a sensor that detects the user's pulse, blood oxygen concentration, brain waves, respiration, body temperature, or the like. The physical condition sensor 14A may be a sensor that detects that the user has pressed a predetermined button. The predetermined button is, for example, a button for the user to notify the server 30 of the deterioration of his/her physical condition. This button may be a button displayed on a touch-up display.

When the bed unit 10A is connected to the traveling unit 10B, electric power may supplied from the traveling unit 10B to the bed unit 10A, and when the bed unit 10A is connected to the house 200, electric power may be supplied from the house 200 to the bed unit 10A. When the bed unit 10A is connected to both the house 200 and the traveling unit 10B, electric power may be supplied from the house 200 to the traveling unit 10B. The bed unit 10A may be equipped with a mechanism for connection to the house 200. For example, as shown in FIG. 3, when the bed unit 10A is disconnected from the traveling unit 10B, the bed unit 10A may be supported by legs 150. Further, the house 200 may be equipped with a mechanism for connection between the bed unit 10A and the house 200. The method of connecting the bed unit 10A and the house 200 is not limited.

### Functional Configuration: Server

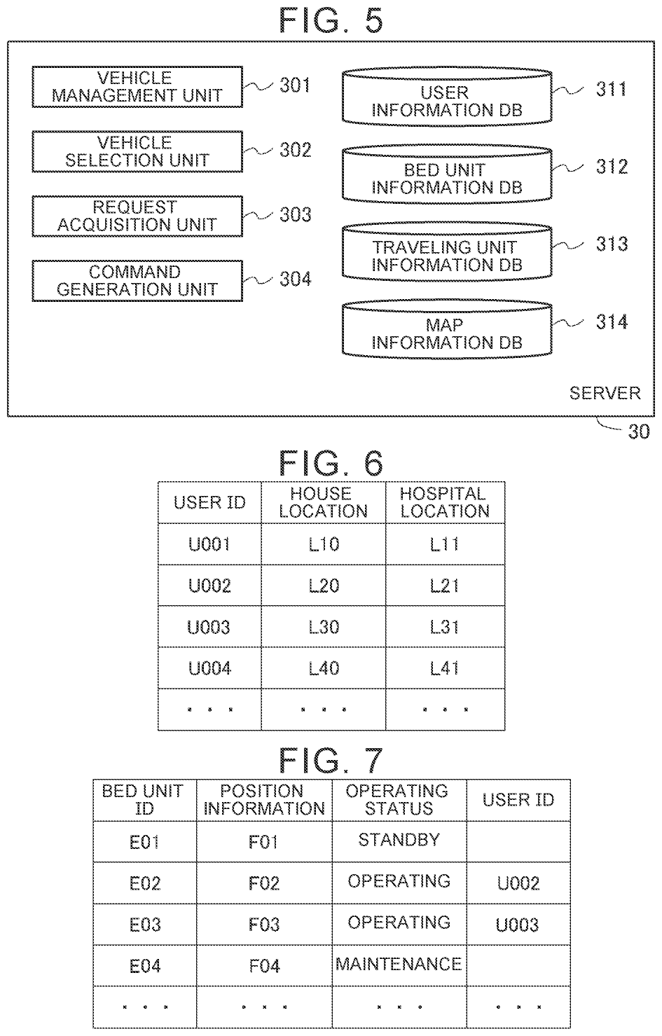

FIG. 5 is a diagram showing an example of a functional configuration of the server 30. The server 30 includes a vehicle management unit 301, a vehicle selection unit 302, a request acquisition unit 303, a command generation unit 304, a user information database (DB) 311, a bed unit information DB 312, a traveling unit information DB 313, and a map information DB 314 as functional components. The vehicle management unit 301, the vehicle selection unit 302, the request acquisition unit 303, and the command generation unit 304 are functional components that are provided, for example, by the processor 31 of the server 30 executing various kinds of programs stored in the auxiliary storage unit 33.

The user information DB 311, the bed unit information DB 312, the traveling unit information DB 313, and the map information DB 314 are, for example, relational databases, which are constructed by the program of a database management system (DBMS) managing the data stored in the auxiliary storage unit 33. The program of the DBMS is executed by the processor 31. Note that any of the functional components of the server 30 or part of the processes thereof may be executed by another computer connected to the network N1.

The vehicle management unit 301 manages various kinds of information regarding the bed unit 10A and the traveling unit 10B. The vehicle management unit 301 manages, for example, the position of the bed unit 10A, the operating status of the bed unit 10A, the position of the traveling unit 10B, and the operating status of the traveling unit 10B. The vehicle management unit 301 acquires and manages the position information transmitted from the bed unit 10A in a predetermined cycle or the position information transmitted from the bed unit 10A in response to a request from the server 30, for example. Further, the vehicle management unit 301 acquires and manages the position information transmitted from the traveling unit 10B in a predetermined cycle or the position information transmitted from the traveling unit 10B in response to a request from the server 30, for example. When the bed unit 10A is connected to the traveling unit 10B, the position information of the connected bed unit 10A may be treated as the same as the position information of the traveling unit 10B. Further, in the case where the bed unit 10A is disconnected from the traveling unit 10B, the position information of the bed unit 10A may be treated as the same as the position information of the traveling unit 10B at the time when the traveling unit 10B disconnects the bed unit 10A. Thus, the position information sensor 17A of the bed unit 10A can be omitted. The vehicle management unit 301 stores in the bed unit information DB 312 the position information of the bed unit 10A in association with a bed unit ID of the bed unit 10A. The bed unit ID is an identifier unique to the bed unit 10A. The vehicle management unit 301 also stores in the traveling unit information DB 313 the position information of the traveling unit 10B in association with the traveling unit ID. The traveling unit ID is an identifier unique to the traveling unit 10B.

Further, the vehicle management unit 301 manages the operating statuses of the bed unit 10A and the traveling unit 10B. The operating status of the bed unit 10A is information for determining the status of the bed unit 10A. The operating status of the bed unit 10A is divided into two states, that is, a standby state and an operating state. For example, when a signal requesting the use of the bed unit 10A is received from the user's terminal, the bed unit 10A in the standby state is offered to the user, so that the bed unit 10A turns to the operating state.

The operating status of the traveling unit 10B is information for determining the status of the traveling unit 10B. The operating status of the traveling unit 10B is divided into two states, that is, a standby state and an operating state. For example, when the traveling unit 10B is carrying the bed unit 10A to the destination, or when the traveling unit 10B is scheduled to carry the bed unit 10A to the destination, the traveling unit 10B is in the operating state. When the traveling unit 10B is not carrying the bed unit 10A and is not scheduled to carry the bed unit 10A, the traveling unit 10B is in the standby state.

For example, when the server 30 transmits to the traveling unit 10B a command to carry the bed unit 10A, the traveling unit 10B turns to the operating state. The operating state includes when the bed unit 10A is placed at the house 200, when the bed unit 10A is moved from the house 200 to the hospital, and when the bed unit 10A is moved from the hospital to the house 200. Further, when a signal indicating that the traveling unit 10B has placed the bed unit 10A at the house 200 is transmitted from the traveling unit 10B to the server 30, or when a signal indicating that the traveling unit 10B has placed the bed unit 10A at the hospital is transmitted from the traveling unit 10B to the server 30, the traveling unit 10B turns to the standby state.

The vehicle management unit 301 stores in the bed information DB 312 the operating status of the bed unit 10A in association with the bed unit ID, and stores in the traveling unit information DB 313 the operating status of the traveling unit 10B in association with the traveling unit ID.

The vehicle selection unit 302 selects the bed unit 10A to be assigned to the user from the plurality of bed units 10A in the standby state, for example. The vehicle selection unit 302 selects, for example, the bed unit 10A closest to the house 200. The vehicle selection unit 302 selects, for example, the traveling unit 10B for carrying the bed unit 10A, from the traveling units 10B in the standby state. The vehicle selection unit 302 selects, for example, the traveling unit 10B for carrying the selected bed unit 10A to the house 200. Further, the vehicle selection unit 302 selects, for example, the traveling unit 10B for moving the bed unit 10A carrying the user from the house 200 to the hospital. At this time, the vehicle selection unit 302 selects the traveling unit 10B for carrying the bed unit 10A from the traveling units 10B located within a predetermined distance from the bed unit 10A. When there is a plurality of traveling units 10B in the standby state within the predetermined distance, the traveling unit 10B, current position of which is closest to the bed unit 10A, may be selected, or the traveling unit 10B may be randomly selected, for example. When the traveling unit 10B is electrically driven, the traveling unit 10B having the highest battery charging rate may be selected. Moreover, the traveling unit 10B with the shortest travel distance or the shortest traveling time may be selected. The predetermined distance may be, for example, a moving distance within an allowable range. The traveling unit 10B may be selected in the same manner, when moving the bed unit 10A from the hospital to the house 200.

The request acquisition unit 303 acquires, for example, the use request transmitted from the bed unit 10A. The use request is information for requesting the use of the traveling unit 10B to move the bed unit 10A. The use request includes, for example, the position information of the bed unit 10A. The use request is generated in the bed unit 10A based on the detection value of the physical condition sensor 14A, and is transmitted from the bed unit 10A to the server 30. Acquiring the use request includes acquiring information regarding deterioration of the physical condition of the user or acquiring information regarding a request from the user for moving the bed unit 10A from the house 200 to the hospital.

The command generation unit 304 generates, for example, an operation command such that the traveling unit 10B starts from the current location, connects to the bed unit 10A placed at the house 200, and then moves to the hospital. The command generation unit 304 generates an operation command such that the traveling unit 10B waits at the hospital or goes away from the hospital after disconnecting the bed unit 10A at the hospital.

The command generation unit 304 transmits the generated operation command to the traveling unit 10B. The command generation unit 304 according to the present embodiment generates a route of the traveling unit 10B based on the map information stored in the map information DB 314 described later and transmits the route to the traveling unit 10B. However, the command generation unit 304 only need to generate information regarding the position of the house 200 (waypoint) and information regarding the position of the hospital (destination). The route in this case is generated in the traveling unit 10B. The route of the traveling unit 10B may be generated so as to be a route pursuant to a predetermined rule, such as a route with the shortest moving distance or a route with the shortest moving time.

The user information DB 311 is provided by storing the user information in the auxiliary storage unit 33. The user information includes, for example, a user ID associated with the user, information regarding the location of the user's house 200, and information regarding the location of the hospital corresponding to the user. Here, the configuration of the user information stored in the user information DB 311 will be described based on FIG. 6. FIG. 6 is a diagram illustrating a table configuration of the user information. A user information table has various fields including a user ID, a location of the house, and a location of the hospital. Information for identifying the user is input to the user ID field. Information regarding the location of the user's house 200 is input in the house location field. The location of the user's house 200 may be a location registered in advance by the user. To the hospital location field, information regarding the location of the hospital corresponding to the user is input. The location of the hospital corresponding to the user may be registered in advance by the user, or may be determined by the command generation unit 304 based on the detection value of the physical condition sensor 14A.

The bed unit information DB 312 is provided by storing information regarding the bed unit 10A (hereinafter, also referred to as “bed unit information”) in the auxiliary storage unit 33. Here, the configuration of the bed unit information stored in the bed unit information DB 312 will be described based on FIG. 7. FIG. 7 is a diagram illustrating a table configuration of the bed unit information. The bed unit information table has various fields including a bed unit ID, the position information, the operating status, and the user ID. Identification information for identifying the bed unit 10A is input to the bed unit ID field. The position information of the bed unit 10A is input to the position information field. This position information is information indicating the current position of the bed unit 10A. When the bed unit 10A is disconnected from the traveling unit 10B, the current position of the bed unit 10A may be fixed until the bed unit 10A is next connected to the traveling unit 10B. To the operating status field, information indicating the status of the bed unit 10A is input. To the operating status field, either “standby” indicating a state in which the bed unit 10A is waiting at a predetermined location or the like, or “operating” indicating a state in which the bed unit 10A is reserved by the user or is being used by the user is input. The user ID of the user using the bed unit 10A is input to the user ID field.

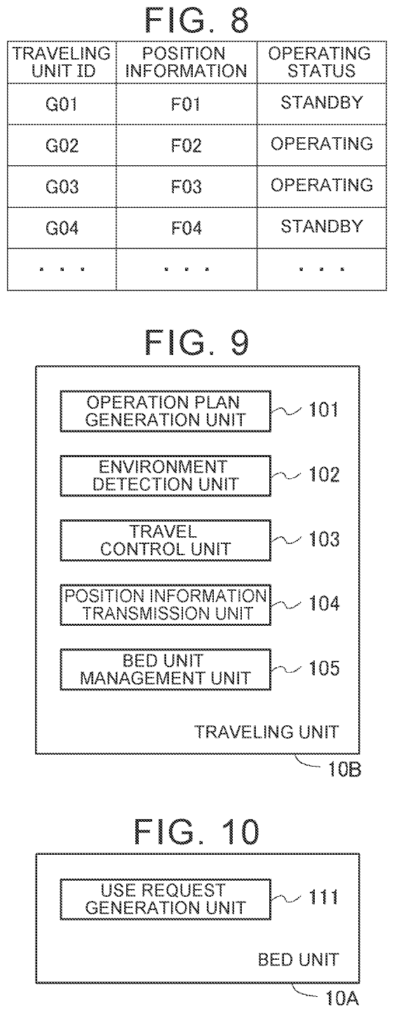

The traveling unit information DB 313 is provided by storing the information regarding the traveling unit 10B (hereinafter, also referred to as “traveling unit information”) in the auxiliary storage unit 33. Here, the configuration of the traveling unit information stored in the traveling unit information DB 313 will be described based on FIG. 8. FIG. 8 is a diagram illustrating a table configuration of the traveling unit information. The traveling unit information table has various fields including a traveling unit ID, the position information, and the operating status. Information for identifying the traveling unit 10B is input to the traveling unit ID field. The position information of the traveling unit 10B is input to the position information field. This position information is information indicating the current position of the traveling unit 10B. To the operating status field, information indicating the status of the traveling unit 10B is input. To the operating status field, either “operating” indicating a state in which the traveling unit 10B is carrying the bed unit 10A to the destination, or a state in which the traveling unit 10B is scheduled to carry the bed unit 10A to the destination, or “standby” indicating a state in which the traveling unit 10B is not carrying the bed unit 10A and is not scheduled to carry the bed unit 10A is input. The state in which the traveling unit 10B is not scheduled to carry the bed unit 10A is, for example, a state in which there is no operation command for carrying the bed unit 10A. The operating status may be transmitted to the server 30 by the traveling unit 10B, or may be determined by the server 30 based on the current position of the traveling unit 10B.

The map information DB 314 stores map data and map information including point-of-interest (POI) information such as characters, photographs, etc. indicating the characteristics of each point on the map data. The map information DB 314 may be provided from another system connected to the network N1, for example, a geographic information system (GIS).

### Functional Structure: Traveling Unit

FIG. 9 is a diagram showing an example of a functional configuration of the traveling unit 10B. The traveling unit 10B includes an operation plan generation unit 101, an environment detection unit 102, a travel control unit 103, a position information transmission unit 104, and a bed unit management unit 105 as functional components. The operation plan generation unit 101, the environment detection unit 102, the travel control unit 103, the position information transmission unit 104, and the bed unit management unit 105 are, for example, functional components that are provided, for example, by the processor 11B of the traveling unit 10B executing various kinds of programs stored in the auxiliary storage unit 13B.

The operation plan generation unit 101 acquires an operation command from the server 30 and generates its operation plan. The operation command includes information regarding a waypoint that the traveling unit 10B passes and a destination. The information regarding the waypoint may include information regarding the location of the house 200, which is the point where the bed unit 10A is connected. Further, the information regarding the destination may include information regarding the location of the hospital, which is the point where the bed unit 10A is disconnected. The operation plan generation unit 101 calculates the route of the traveling unit 10B based on the operation command provided by the server 30 and generates the operation plan for moving on the route.

The environment detection unit 102 detects the environment around the traveling unit 10B that is necessary for autonomous traveling, based on the data acquired by the environment information sensor 18B. Objects to be detected include, for example, the number and the positions of lanes, the number and the positions of other moving bodies around the traveling unit 10B, the number and the positions of obstacles around the traveling unit 10B (pedestrians, bicycles, structures, buildings, etc.), the structure of the road, road signs, and the like, but not limited to these. Any object may be detected as long as it is necessary for autonomous traveling. For example, when the environment information sensor 18B is a stereo camera, image data taken thereby is subjected to image processing to detect the objects around the traveling unit 10B. Data about the surrounding environment of the traveling unit 10B (hereinafter, environmental data) detected by the environment detection unit 102 is transmitted to the travel control unit 103 described later.

The travel control unit 103 generates the control command for controlling autonomous traveling of the traveling unit 10B, based on the operation plan generated by the operation plan generation unit 101, the environmental data generated by the environment detection unit 102, and the position information of the traveling unit 10B that is acquired by the position information sensor 17B. For example, the travel control unit 103 generates the control command to cause the traveling unit 10B to travel along a predetermined route while suppressing obstacles from entering a predetermined safety area around traveling unit 10B. The generated control command is transmitted to the drive unit 19B. A known method can be adopted as a method of generating a control command for causing the traveling unit 10B to travel autonomously.

The position information transmission unit 104 transmits the position information acquired from the position information sensor 17B to the server 30 via the communication unit 16B. The timing at which the position information transmission unit 104 transmits the position information can be set as appropriate, and for example, the position information may be transmitted periodically, may be transmitted in synchronization with the timing of transmitting some information to the server 30, or may be transmitted in response to a request from the server 30. The position information transmission unit 104 transmits the position information to the server 30 together with the traveling unit ID.

The bed unit management unit 105 generates a command regarding connection and disconnection of the bed unit 10A and the traveling unit 10B. The command includes a command for instructing the connection device 14B to connect the bed unit 10A to the traveling unit 10B, a command for instructing the connection device 14B to disconnect the bed unit 10A from the traveling unit 10B, and the like.

### Functional Structure: Bed Unit

FIG. 10 is a diagram showing an example of a functional configuration of the bed unit 10A. The bed unit 10A includes a use request generation unit 111 as a functional component. The use request generation unit 111 is a functional component provided by, for example, the processor 11A of the bed unit 10A executing various kinds of programs stored in the auxiliary storage unit 13A.

The use request generation unit 111 generates a use request (first request) based on the detection value of the physical condition sensor 14A. The use request generation unit 111 generates a use request when the detection value of the physical condition sensor 14A has a value at which it is desirable to transport the user to the hospital. In addition, the use request generation unit 111 generates a use request when the physical condition sensor 14A detects that the user desires to be transported to the hospital. The use request includes the bed unit ID and the position information of the bed unit 10A. The use request may include the detection value of the physical condition sensor 14A. The server 30 may use the detection value to determine the hospital to which the user is transported. Further, the server 30 may transmit the detection value of the physical condition sensor 14A to the terminal of the hospital in advance. After generating the use request, the use request generation unit 111 transmits the use request to the server 30. Note that the use request generation unit 111 may transmit the detection value of the physical condition sensor 14A to the server 30 even when the use request generation unit 111 does not generate the use request. Then, the server 30 may record the detection value of the physical condition sensor 14A. In the case where a push button or a touch-up display is provided as the physical condition sensor 14A, the use request generation unit 111 may generate the use request when the user presses the push button or taps a predetermined point on the touch-up display.

### Process Flow: System

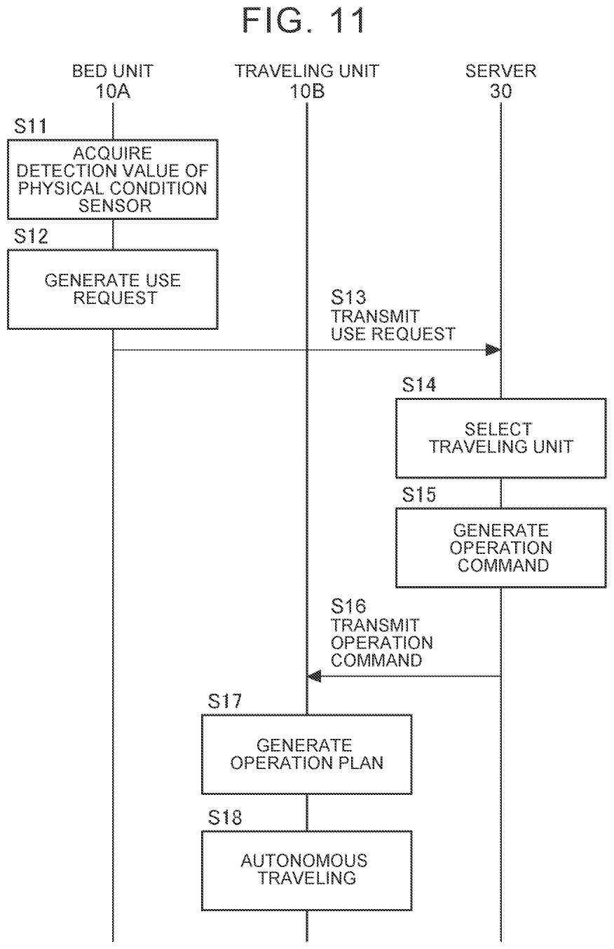

Next, overall processes of the autonomous driving system 1 will be described. FIG. 11 is a sequence diagram showing processes of the autonomous driving system 1. In FIG. 11, it is assumed that the position information is transmitted from the bed unit 10A and the traveling unit 10B as needed. Further, it is assumed that the bed unit 10A is connected to the house 200 and disconnected from the traveling unit 10B. Further, it is assumed that the user is in the bed unit 10A.

In the bed unit 10A, the detection value of the physical condition sensor 14A is acquired (511), and the use request is generated based on the detection value of the physical condition sensor 14A (S12). When the use request is generated in the bed unit 10A, the use request is transmitted from the bed unit 10A to the server 30 (S13). The server 30 having received the use request selects the traveling unit 10B for moving the bed unit 10A (S14). At this time, the traveling unit 10B located within a predetermined distance from the bed unit 10A is selected. Then, the server 30 generates an operation command based on the current positions of the bed unit 10A and the traveling unit 10B (S15), and transmits the operation command to the traveling unit 10B (S16). The traveling unit 10B that has received the operation command generates the operation plan (S17), and the traveling unit 10B performs autonomous traveling based on the operation plan (S18).

### Process Flow: Server

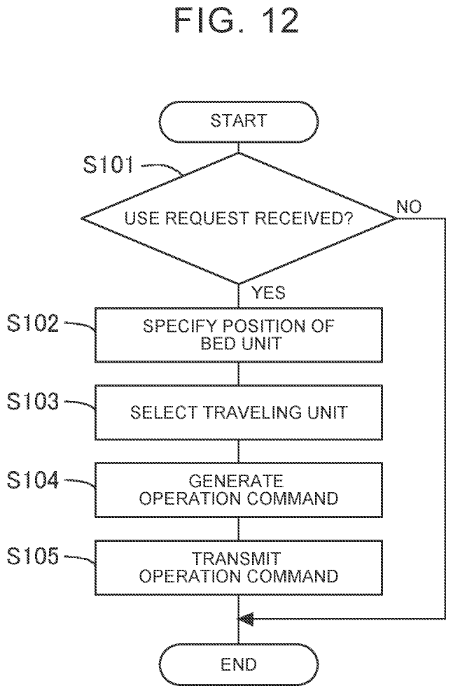

Next, processes in the server 30 will be described. FIG. 12 is an example of a flowchart illustrating processes in the server 30 according to the present embodiment. The processes shown in FIG. 12 are executed by the processor 31 of the server 30 at predetermined cycles. Here, it is assumed that the server 30 has already received the information necessary for constructing the bed unit information DB 312 and the traveling unit information DB 313.

In step S101, it is determined whether the request acquisition unit 303 has received a use request from the bed unit 10A. The use request includes the bed unit ID and the position information of the bed unit 10A. When an affirmative determination is made in step S101, the process proceeds to step S102, and when a negative determination is made, the routine is terminated.

In step S102, the vehicle selection unit 302 specifies the position of the bed unit 10A. The vehicle selection unit 302 accesses the bed unit information DB 312 and acquires the position information of the bed unit 10A to specify the position of the bed unit 10A.

In step S103, the vehicle selection unit 302 selects the traveling unit 10B for carrying the bed unit 10A that has transmitted the use request (first traveling unit). The vehicle selection unit 302 accesses the traveling unit information DB 313 and the map information DB 314 and selects one traveling unit 10B located within the predetermined distance from the bed unit 10A among the traveling units 10B with their operating status being “standby”. At this time, the traveling unit 10B with the shortest distance from the bed unit 10A may be selected. The predetermined distance is determined, for example, so that the time required for the traveling unit 10B to arrive at the current position of the bed unit 10A falls within an allowable range. When there is no traveling unit 10B within the predetermined distance from the bed unit 10A, the process may wait for the traveling unit 10B in the operating state to turn to the standby state, or the traveling unit 10B outside the predetermined distance from the bed unit 10A may be selected.

In step S104, the command generation unit 304 generates an operation command to be transmitted to the traveling unit 10B. The command generation unit 304 generates an operation command including the following; that is, the traveling unit 10B moves from the current position of the traveling unit 10B to the current position of the bed unit 10A, the traveling unit 10B connects the bed unit 10A at the current position of the bed unit 10A (that is, the location of the house 200), the traveling unit 10B moves from the current position of the bed unit 10A to the hospital, and the traveling unit 10B disconnects the bed unit 10A at the hospital. After the command generation unit 304 generates the operation command, the process proceeds to step S105, where the operation command is transmitted to the traveling unit 10B selected in step S103. At this time, the operating status field of the traveling unit 10B in the traveling unit information DB 313 is updated from “standby” to “operating”.

### Process Flow: Traveling Unit

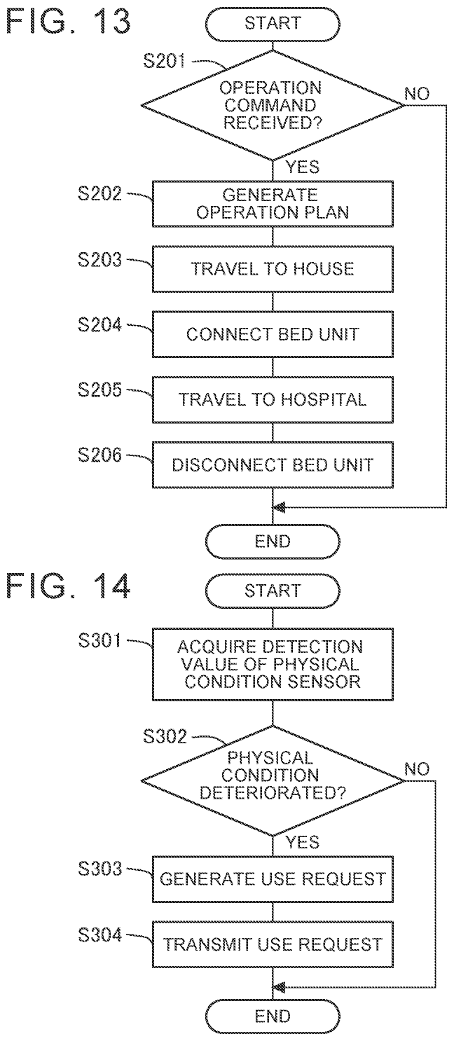

Next, processes in the traveling unit 10B will be described. FIG. 13 is an example of a flowchart illustrating processes in the traveling unit 10B according to the present embodiment. The processes shown in FIG. 13 are executed by the processor 11B of the traveling unit 10B at predetermined cycles. The flowchart is implemented by the traveling unit 10B in the standby state.

In step S201, it is determined whether the operation plan generation unit 101 has received an operation command from the server 30. When an affirmative determination is made in step S201, the process proceeds to step S202, and when a negative determination is made, the routine is terminated. In step S202, the operation plan generation unit 101 generates an operation plan in accordance with the operation command.

When the generation of the operation plan is completed, in step S203, the travel control unit 103 generates a control command. The drive unit 19B is controlled in accordance with the control command, and the traveling unit 10B travels to the user's house 200. The bed unit 10A carrying the user is placed at the house 200. When the traveling unit 10B arrives at the house 200, the process proceeds to step S204, and the bed unit management unit 105 controls the connection device 14B such that the bed unit 10A is connected to the traveling unit 10B.

When the connection of the bed unit 10A is completed, the process proceeds to step S205, the travel control unit 103 generates a control command. The drive unit 19B is controlled in accordance with the control command, and the traveling unit 10B travels to the hospital. When the traveling unit 10B arrives at the hospital, the process proceeds to step S206, and the bed unit management unit 105 disconnects the bed unit 10A from the traveling unit 10B and ends this routine. After that, the server 30 may transmit a command to wait at the hospital or a command to move to a predetermined place to the traveling unit 10B.

### Process Flow: Bed Unit

Next, processes of transmitting the use request in the bed unit 10A will be described. FIG. 14 is an example of a flowchart of processes of transmitting a use request according to the present embodiment. The processes shown in FIG. 14 are executed at predetermined cycles by the processor 11A of the bed unit 10A placed at the house 200.

In step S301, the use request generation unit 111 acquires the detection value of the physical condition sensor 14A. In step S302, the use request generation unit 111 determines whether the physical condition of the user has deteriorated, based on the detection value of the physical condition sensor 14A. In step S302, the use request generation unit 111 determines whether the detection value of the physical condition sensor 14A is a value at which it is desirable to transport the user to the hospital. In step S302, the use request generation unit 111 may determine that the physical condition of the user has deteriorated when the push button provided as the physical condition sensor 14A is pressed or when a predetermined point on the touch-up display provided as the physical condition sensor 14A is tapped. When an affirmative determination is made in step S302, the process proceeds to step S303, and when a negative determination is made, the routine is terminated. Next, in step S303, the use request generation unit 111 generates a use request. Then, in step S304, the use request generation unit 111 transmits the use request to the server 30.

As described above, according to the present embodiment, the bed unit 10A can be provided to the user by using the autonomously traveling vehicle 10, and the user can be carried together with the bed unit 10A from the house 200 to the hospital. Thus, even when the user has difficulty walking, the user can be quickly transported to the hospital. Further, since the traveling unit 10B for carrying the bed unit 10A is selected from the traveling units 10B located within a predetermined distance from the bed unit 10A, the traveling unit 10B can quickly arrive at the place where the bed unit 10A is placed. Further, since the traveling unit 10B can carry another bed unit 10A after separating the bed unit 10A, the operating rate of the traveling unit 10B can be increased.

### Other Embodiments

The above-described embodiment is merely an example, and the present disclosure may be appropriately modified and implemented without departing from the scope thereof.

The processes and means described in the present disclosure can be freely combined and implemented as long as no technical contradiction occurs.

Further, the processes described as being executed by one device may be shared and executed by a plurality of devices. Alternatively, the processes described as being executed by different devices may be executed by one device. In the computer system, it is possible to flexibly change the hardware configuration (server configuration) for realizing each function. Although in the above-described embodiment, the server 30 includes, as functional components, the vehicle management unit 301, the vehicle selection unit 302, the request acquisition unit 303, the command generation unit 304, the user information DB 311, the bed unit information DB 312, the traveling unit information DB 313, and the map information DB 314, part or all of these functional components may be included in the bed unit 10A or the traveling unit 10B.

Moreover, in the above embodiment, the case where the bed unit 10A is carried from the house 200 to the hospital is described. The case where the bed unit 10A is carried from the hospital to the house 200 can be considered in the same manner. In this case, for example, the user or a hospital staff (staff including doctors and nurses) inputs a request for carrying the bed unit 10A from the hospital to the house 200 (second request) to a terminal of the hospital or the input unit included in the bed unit 10A. A use request is generated in accordance with the input and is transmitted to the server 30. When the server 30 receives this use request, the server 30 selects the traveling unit 10B located within a predetermined distance from the bed unit 10A (second traveling unit) and generates the operation command for the traveling unit 10B. The command generation unit 304 generates an operation command including the following; that is, the traveling unit 10B moves from the current position of the traveling unit 10B to the current position of the bed unit 10A, the traveling unit 10B connects the bed unit 10A at the current position of the bed unit 10A (that is, the location of the hospital), the traveling unit 10B moves from the current position of the bed unit 10A to the house 200, and the traveling unit 10B disconnects the bed unit 10A at the house 200. Then, the operation command is transmitted to the selected traveling unit 10B.

The processes will be described with reference to FIG. 12. Matters different from those described in the first embodiment will be mainly described. In step S101, it is determined whether the request acquisition unit 303 has received a use request from the bed unit 10A. The use request mentioned here is a request to carry the bed unit 10A from the hospital to the house 200, and is generated, for example, in the bed unit 10A or the terminal of the hospital. This use request includes the bed unit ID and the position information of the bed unit 10A.

In step S102, the vehicle selection unit 302 specifies the position of the bed unit 10A. The vehicle selection unit 302 accesses the bed unit information DB 312 and acquires the position information of the bed unit 10A to specify the position of the bed unit 10A.

In step S103, the vehicle selection unit 302 selects the traveling unit 10B for carrying the bed unit 10A (second traveling unit). The vehicle selection unit 302 accesses the traveling unit information DB 313 and the map information DB 314 and selects one traveling unit 10B located within the predetermined distance from the bed unit 10A among the traveling units 10B with their operating status being “standby”. At this time, the traveling unit 10B with the shortest distance from the bed unit 10A may be selected. The predetermined distance is determined, for example, so that the time required for the traveling unit 10B to arrive at the current position of the bed unit 10A falls within an allowable range. When there is no traveling unit 10B within the predetermined distance from the bed unit 10A, the process may wait for the traveling unit 10B in the operating state to turn to the standby state, or the traveling unit 10B outside the predetermined distance from the bed unit 10A may be selected. The predetermined distance may have a value different from that in the first embodiment. That is, it is considered that the movement from the hospital to the house 200 is less urgent than the movement from the house 200 to the hospital, and thus a longer traveling time may be allowed for the traveling unit 10B. Thus, in the case of movement from the hospital to the house 200, the traveling unit 10B may be selected from a wider range.

In step S104, the command generation unit 304 generates an operation command to be transmitted to the traveling unit 10B. The command generation unit 304 generates an operation command including the following; that is, the traveling unit 10B moves from the current position of the traveling unit 10B to the current position of the bed unit 10A, the traveling unit 10B connects the bed unit 10A at the current position of the bed unit 10A (that is, the location of the hospital), the traveling unit 10B moves from the current position of the bed unit 10A to the house 200, and the traveling unit 10B disconnects the bed unit 10A at the house 200. After the command generation unit 304 generates the operation command, the process proceeds to step S105, where the operation command is transmitted to the traveling unit 10B selected in step S103. At this time, the operating status field of the traveling unit 10B in the traveling unit information DB 313 is updated from “standby” to “operating”.

The present disclosure can also be implemented by supplying a computer with a computer program that implements the functions described in the above embodiments, and causing one or more processors of the computer to read and execute the program. Such a computer program may be provided to the computer by a non-transitory computer-readable storage medium connectable to the system bus of the computer, or may be provided to the computer via a network. The non-transitory computer-readable storage medium is, for example, a disc of any type such as a magnetic disc (floppy (registered trademark) disc, hard disk drive (HDD), etc.), an optical disc (compact disc (CD)-ROM, digital versatile disc (DVD), Blu-ray disc, etc.), a read only memory (ROM), a random access memory (RAM), an erasable programmable read only memory (EPROM), an electrically erasable programmable read only memory (EEPROM), a magnetic card, a flash memory, an optical card, and any type of medium suitable for storing electronic commands.

What is claimed is:

1. An information processing device comprising a control unit that executes:
acquiring a first request that is a request based on information regarding a physical condition of a user using a bed unit that functions as a bed for the user, and that is a request to move the bed unit from a house of the user to a hospital;
when having acquired the first request, selecting a first traveling unit that is a traveling unit that moves the bed unit from the house to the hospital and that is a traveling unit located within a predetermined distance from the bed unit; and
generating a command for the first traveling unit such that the first traveling unit carries the bed unit from the house to the hospital.

2. The information processing device according to claim 1, wherein acquiring the first request includes acquiring information regarding deterioration of the physical condition of the user.

3. The information processing device according to claim 2, wherein the information regarding the deterioration of the physical condition of the user includes information regarding a detection value of a sensor that measures a physical quantity related to the physical condition of the user.

4. The information processing device according to claim 1, wherein acquiring the first request includes acquiring information regarding a request from the user to move the bed unit from the house to the hospital.

5. The information processing device according to claim 1, wherein the control unit selects a traveling unit located closest to the bed unit when selecting the first traveling unit.

6. The information processing device according to claim 1, wherein the command for the first traveling unit includes a command to disconnect the bed unit after carrying the bed unit to the hospital.

7. The information processing device according to claim 1, wherein the control unit executes:
acquiring a second request to carry the bed unit from the hospital to the house;
when having acquired the second request, selecting a second traveling unit that is a traveling unit that moves the bed unit from the hospital to the house and that is a traveling unit located within the predetermined distance from the bed unit; and
generating a command for the second traveling unit such that the second traveling unit carries the bed unit from the hospital to the house.

8. The information processing device according to claim 7, wherein the command for the second traveling unit includes a command to disconnect the bed unit after carrying the bed unit to the house.

9. An information processing method for causing a computer to execute:
acquiring a first request that is a request based on information regarding a physical condition of a user using a bed unit that functions as a bed for the user, and that is a request to move the bed unit from a house of the user to a hospital;
when having acquired the first request, selecting a first traveling unit that is a traveling unit that moves the bed unit from the house to the hospital and that is a traveling unit located within a predetermined distance from the bed unit; and
generating a command for the first traveling unit such that the first traveling unit carries the bed unit from the house to the hospital.

10. The information processing method according to claim 9, wherein acquiring the first request includes acquiring information regarding deterioration of the physical condition of the user.

11. The information processing method according to claim 10, wherein the information regarding the deterioration of the physical condition of the user includes information regarding a detection value of a sensor that measures a physical quantity related to the physical condition of the user.

12. The information processing method according to claim 9, wherein acquiring the first request includes acquiring information regarding a request from the user to move the bed unit from the house to the hospital.

13. The information processing method according to claim 9, wherein when the first traveling unit is selected, a traveling unit located closest to the bed unit is selected.

14. The information processing method according to claim 9, wherein the command for the first traveling unit includes a command to disconnect the bed unit after carrying the bed unit to the hospital.

15. The information processing method according to claim 9, the method executing:
acquiring a second request to carry the bed unit from the hospital to the house;
when having acquired the second request, selecting a second traveling unit that is a traveling unit that moves the bed unit from the hospital to the house and that is a traveling unit located within the predetermined distance from the bed unit; and
generating a command for the second traveling unit such that the second traveling unit carries the bed unit from the hospital to the house.

16. The information processing method according to claim 15, wherein the command for the second traveling unit includes a command to disconnect the bed unit after carrying the bed unit to the house.

17. A system comprising:
a bed unit that functions as a bed for a user;
a traveling unit that is connected to the bed unit and that moves the bed unit; and
a server having a control unit that controls the traveling unit, wherein the control unit executes:
acquiring a first request that is a request based on information regarding a physical condition of the user using the bed unit and that is a request to move the bed unit from a house of the user to a hospital;
when having acquired the first request, selecting a first traveling unit that is a traveling unit that moves the bed unit from the house to the hospital and that is a traveling unit located within a predetermined distance from the bed unit; and
generating a command for the first traveling unit such that the first traveling unit carries the bed unit from the house to the hospital.

18. The system according to claim 17, wherein acquiring the first request includes acquiring information regarding deterioration of the physical condition of the user or acquiring information regarding a request from the user to move the bed unit from the house to the hospital.

19. The system according to claim 17, wherein the command for the first traveling unit includes a command to leave the hospital after carrying the bed unit to the hospital.

20. The system according to claim 17, wherein the control unit executes:
acquiring a second request to carry the bed unit from the hospital to the house;
when having acquired the second request, selecting a second traveling unit that is a traveling unit that moves the bed unit from the hospital to the house and that is a traveling unit located within the predetermined distance from the bed unit; and
generating a command for the second traveling unit such that the second traveling unit carries the bed unit from the hospital to the house.
